(19) United States
(12) Patent Application Publication
(10) Pub. No.: US 2024/0341615 A1
(43) Pub. Date: Oct. 17, 2024

(54) Needle Assembly with Fluid Bypassing Stylet

(71) Applicant: Bard Access Systems, Inc., Salt Lake City, UT (US)

(72) Inventor: Ming F. Hsu, Wayne, NJ (US)

(21) Appl. No.: 18/135,353

(22) Filed: Apr. 17, 2023

(51) Int. Cl. A61B 5/0538; A61B 5/15

(52) U.S. Cl. CPC A61B 5/0538; A61B 5/15003; A61B 5/150511; A61B 5/150519

(57) ABSTRACT

A medical needle assembly includes a stylet having non-circular cross-sectional shape disposed within the needle lumen. The needle and the stylet each define respective electrodes that enable ascertainment of an electrical property of a body substance adjacent the distal tip of the needle. An electrically insulative coating having a uniform thickness covers the stylet. Wire leads enable connection of the electrodes to a measurement module configured to measure an electrical property of a body substance adjacent a distal end of the needle during insertion. Electrical property measurement enables identification of the location of the needle tip within the patient based on the measurement value. A major diameter of the non-circular cross-sectional shape extends across the needle lumen to create first and second flow paths extending along the lumen on opposite sides of non-circular cross-sectional shape. The first and second flow paths enable blood to flow along the lumen during use.

### BACKGROUND

In some instances, accessing a vasculature with a needle may include complications such as misplacement of the needle. Such misplacement may cause harm to the patient and require additional time to properly place the needle adding cost to the procedure. A common procedure is to obtain a blood flash by drawing blood through the needle lumen to verify that the needle tip is placed within the blood vessel. Some needles may include a stylet inserted within the lumen of the needle creating a blockage of the needle lumen that prevents obtaining a blood flash.

Disclosed herein are systems, devices, and method that address the forgoing.

### SUMMARY

Disclosed herein is a medical needle assembly that, according to some embodiments, includes a needle configured for vascular access of a patient, where the needle defines a needle lumen extending between a proximal end and a distal end of the needle. The assembly further includes a stylet disposed within the needle lumen, where the stylet includes a non-circular cross-sectional shape. The needle defines a needle electrode, and the stylet includes a stylet electrode adjacent a distal end of the stylet. The needle and stylet electrodes are configured to enable ascertainment of an electrical property of a body substance adjacent the distal end of the needle.

In some embodiments, the stylet is formed of a stainless steel, and in such embodiments, the stylet includes an electrically insulative coating extending along a length of the stylet. In some embodiments, the coating defines a uniform thickness.

In some embodiments, the non-circular cross-sectional shape extends along an entire length of the stylet. In some embodiments, a major diameter of the non-circular cross-sectional shape extends across the needle lumen, and in some embodiments, the needle lumen defines a first flow path extending along a first side of the non-circular cross-sectional shape and a second flow path extending along a second side of the non-circular cross-sectional shape, where the second side is opposite the first side. In some embodiments, the non-circular cross-sectional shape includes an oval.

In some embodiments, the needle includes a needle lead configured to electrically couple the needle electrode to a measurement module, and the stylet includes a stylet lead configured to electrically couple the stylet electrode to the measurement module.

In some embodiments, the needle includes a hub that includes a fluid port in fluid communication with at least one of the first or second flow paths and a stylet port configured to receive the stylet therethrough, where the stylet port defines a fluid tight seal with the non-circular shape. In some embodiments, the stylet is configured for removal from the needle, and the stylet port includes a septum configured to define a fluid tight seal across the stylet port when the stylet is removed from the needle.

Also disclosed herein is a medical stylet that, according to some embodiments, includes a non-circular cross-sectional shape extending along the stylet from a proximal end to the distal end of the stylet and an electrode adjacent the distal end of the stylet, where the electrode is configured to electronically couple with a substance of a patient. The stylet further includes a stylet lead configured to electrically couple the stylet electrode to a measurement module.

In some embodiments, the stylet is formed of a stainless steel, and the stylet includes an electrically insulative coating extending along a length of the stylet, where the coating defines a uniform thickness.

In some embodiments, the stylet is configured for insertion within a lumen of a vascular access needle, and a major diameter of the non-circular shape is extends across the lumen. In such embodiments, the lumen defines a first flow path extending along a first side of the non-circular shape and a second flow path extending along a second side of the non-circular shape, where the second side is opposite the first side.

Also disclosed herein is a method of accessing a vasculature of a patient that, according to some embodiments, includes providing a needle assembly, that includes a needle configured for vascular access of a patient, where the needle defines a needle lumen extending between a proximal end and a distal end of the needle. The needle assembly further includes a stylet disposed within the needle lumen. The stylet includes a non-circular cross-sectional shape. The needle defines a needle electrode, and the stylet includes a stylet electrode adjacent a distal end of the stylet, where the needle and stylet electrodes are configured to enable ascertainment of an electrical property of a body substance adjacent the distal end of the needle. The method further includes (i) inserting the needle to first location within the patient; (ii) obtaining a first value of an electrical property between the needle and stylet electrodes, where the first value is defined by a first body substance located at the first location; and (iii) determining the first location based on the first value of the electrical property.

In some embodiments of the method, the stylet is formed of a stainless steel and the stylet includes an electrically insulative coating extending along a length of the stylet, where the coating defines a uniform thickness.

In some embodiments, the method further includes (i) inserting the needle to a second location within the patient, where the second location is different from the first location; (ii) obtaining a second value of the electrical property between the needle and stylet electrodes, where the second value is defined by a second body substance located at the second location; and (iii) determining the second location based on the second value of the electrical property.

In some embodiments of the method, the non-circular cross-sectional shape defines a flow path extending along the needle lumen.

In some embodiments of the method, the needle includes a hub, having a fluid port in fluid communication with the flow path and a stylet port configured to receive the stylet therethrough, where the stylet port defines a fluid tight seal with the non-circular shape.

In some embodiments of the method, the fluid port is coupled with a syringe via a tube, and the method further includes drawing blood through the flow path and into the tube and observing blood within the tube to confirm placement of the needle within the vasculature.

These and other features of the concepts provided herein will become more apparent to those of skill in the art in view of the accompanying drawings and following description, which describe particular embodiments of such concepts in greater detail.

### DRAWINGS

FIG. 1 illustrates a medical needle assembly including a stylet disposed within the needle lumen, in accordance with some embodiments.

FIG. 2 is a cross-section end view of the medical needle assembly of FIG. 1, in accordance with some embodiments.

FIGS. 3A-3C illustrate a distal portion of the medical needle assembly of FIG. 1 in various stages of insertion within a patient, in accordance with some embodiments.

FIG. 4 is a block diagram of a method of accessing a vasculature utilizing the medical needle assembly of FIG. 1, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. More specifically, the proximal end of a medical device is the end nearest a practitioner during use, and the distal end of a medical device is the end or portion nearest a patient during use. For example, the distal end or portion of a catheter is the end or portion of the catheter furthest disposed within the patient. Conversely, the proximal end or portion of the catheter is the end or portion disposed outside the patient.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

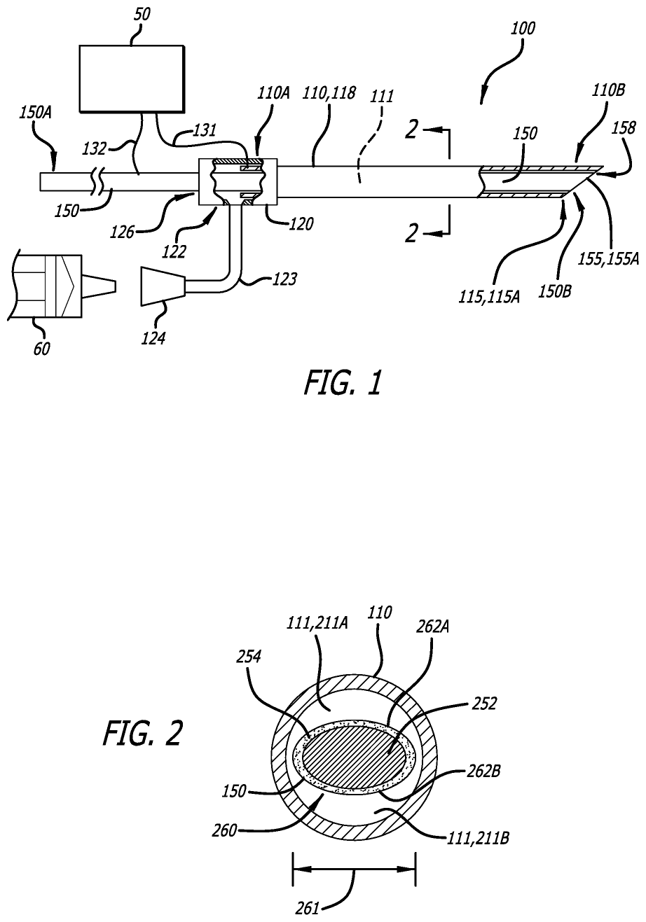

FIG. 1 illustrates a medical needle assembly configured to enable a clinician to access a vasculature of the patient. According to some embodiments, the medical needle assembly (assembly) 100 generally includes a needle 110 configured for insertion into the patient between a skin surface and a vasculature of the patient and a stylet 150 disposed within a needle lumen 111 of the needle 110. The needle lumen 111 extends between a proximal end 110A and a distal end 110B of the needle 110.

The needle 110 defines a beveled needle tip 115 at the distal end 110B and the beveled needle tip 115 defines a bevel surface 115A. The stylet 150 extends between a proximal end 150A and a distal end 150B of the stylet 150. In some embodiments, the stylet 150 may define a beveled stylet tip 155, and the beveled stylet tip 155 may also define a bevel surface 155A. In some embodiments, the bevel surface 115A of the needle 110 may be aligned with the bevel surface 155A of the stylet 150. In other embodiments, the bevel surface 155A may be recessed with respect to the bevel surface 115A or may extend beyond the bevel surface 115A.

The needle 110 may include a hub 120 coupled with the needle 110 at the proximal end 110A, according to some embodiments. The hub 120 may include a fluid port 122 in fluid communication with the needle lumen 111. A tube 123 is coupled with the fluid port 122 such that the tube 123 is in fluid communication with the needle lumen 111. The tube 123 includes a connector 124 configured to couple with a syringe 60. In use, the clinician may utilize the syringe 60 to draw fluid (e.g., blood) along the needle lumen 111 and into the tube 123 to obtain a blood flash.

The needle 110 defines a needle electrode 118 configured to electrically couple with a body substance of the patient when the needle 110 is inserted into the patient. For example, the needle 110 may be formed of a stainless steel to define the electrode 118. The stylet 150 also includes a stylet electrode 158. The stylet electrode 158 is disposed adjacent the distal end 150B of the stylet 150. In some embodiments, the bevel surface 155A of the stylet 150 may define the stylet electrode 158. The stylet electrode 158 is configured to electrically couple with a body substance adjacent the distal end 150B.

The needle 110 includes a needle lead 131 (e.g., a wire) coupled with the needle electrode 118 and configured to couple with a measurement module 50. The stylet 150 also includes a stylet lead 132 coupled with the stylet electrode 158 and configured to couple with the measurement module 50. As such, during use, the measurement module 50 may measure a value of an electrical property of a body substance extending between the needle electrode 118 and the stylet electrode 158 as further described below.

FIG. 2 is a cross-sectional view of the assembly 100 cut along sectioning lines 2-2 showing the stylet 150 disposed within the needle lumen 111. The stylet 150 includes a non-circular cross-sectional shape 260 to provide space for fluid to flow along the needle lumen 111. In some embodiments, the non-circular cross-sectional shape 260 may include an oval. In some embodiments, the non-circular cross-sectional shape 260 may extend along an entire length of the stylet 150 consistent with manufacturing the stylet 150 via an extrusion process.

The non-circular cross-sectional shape 260 includes a major diameter 261 and in some embodiments, the major diameter 261 may extend across the needle lumen 111. In such embodiments, the major diameter 261 may define a first flow path 211A extending along a first side 262A of the stylet 150 (e.g., along the first side 262A of the non-circular cross-sectional shape 260). Similarly, the major diameter 261 may define a second flow path 211B extending along a second side 262B of the stylet 150, where the second side 262B is disposed opposite the first side 262A. Either or both of the first and second flow paths 211A, 211B may be configured for passage of blood therealong.

In some embodiments, the stylet 150 includes a stainless steel core 252 covered by an electrically insulative coating 254. In some embodiments, the coating 254 may be configured to ensure that only the bevel surface 155A of the stylet 150 serves as the electrode 158. The coating 254 may also prevent electrical contact between the stainless steel core 252 and the needle 110. In some embodiments, the coating 254 may extend along an entire length of the stylet 150. In other embodiments, the coating 254 may only extend along a portion (or length) of the stylet 150 disposed within the needle lumen 111. The coating 254 may define a uniform thickness consistent with applying the coating 254 via an extrusion process. In other embodiments, the needle 110 may, in lieu of or in addition to the coating 254, include a coating (not shown) disposed on an inside surface of the needle 110.

Although the stylet 150 is described as a component of the assembly 100, the stylet 150 may also constitute a stand-alone device. In other words, the stylet 150 may be provided separately from the needle 110. As such, the clinician may couple the stylet 150 with the needle 110, such as at a point of use, for example.

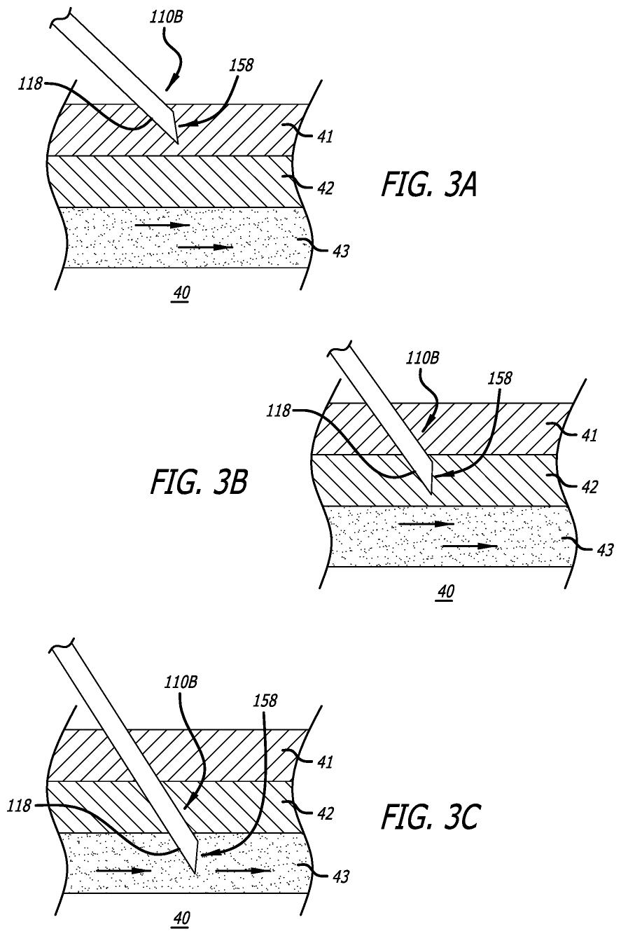

FIGS. 3A-3C illustrate a distal portion of the assembly 100 at various stages of insertion within a patient 40. Shown in FIGS. 3A-3C is cross-sectional view of the patient 40 including layers of body tissue (or substance), such as a skin layer 41, a subdermal tissue layer 42, and a blood vessel 43, for example. Each of the layers 41-43 define different properties, including electrical properties, such as resistivity or conductivity, for example. Such electrical properties (or levels of the electrical properties) may be determined via electrical measurements, such as impedance, capacitance, or inductance measurements, for example. By way of one example, the skin layer 41 may define a first resistivity, the subdermal tissue layer 42 may define a second resistivity, and the blood vessel 43 (or more specifically blood within the blood vessel 43) may define a third resistivity, where the first second and third resistivities are different from each other.

FIG. 3A illustrates a first instance of the example, where the distal end 110B of the needle 110 is disposed within the skin layer 41 such that the needle electrode 118 and the stylet electrode 158 are electrically coupled with the skin layer 41 (or the body substance of the skin layer 41). As such, the first resistivity of the skin layer 41 defines a first impedance between the needle and stylet electrodes 118, 158. In such a first instance, the measurement module 50 (FIG. 1) may measure the first impedance as defined by the first resistivity, and thereby determine or identify that the location of distal end 110B is within the skin layer 41.

FIG. 3B illustrates a second instance of the example where the distal end 110B of the needle 110 is disposed within the subdermal layer 42 such that the needle electrode 118 and the stylet electrode 158 are electrically coupled with the subdermal layer 42 (or the body substance of the subdermal layer 42). As such, the second resistivity of the subdermal layer 42 defines a second impedance between the needle and stylet electrodes 118, 158. In such a second instance, the measurement module 50 (FIG. 1) may measure the second impedance as defined by the second resistivity, and thereby determine or identify that the location of distal end 110B is within the subdermal layer 42.

FIG. 3C illustrates a third instance of the example where the distal end 110B of the needle 110 is disposed within the blood vessel 43 such that the needle electrode 118 and the stylet electrode 158 are electrically coupled with the blood vessel 43 (or the blood within the blood vessel 43). As such, the third resistivity of the blood vessel 43 defines a third impedance between the needle and stylet electrodes 118, 158. In such a third instance, the measurement module 50 (FIG. 1) may measure the third impedance as defined by the third resistivity, and thereby determine or identify that the location of distal end 110B is within the blood vessel 43.

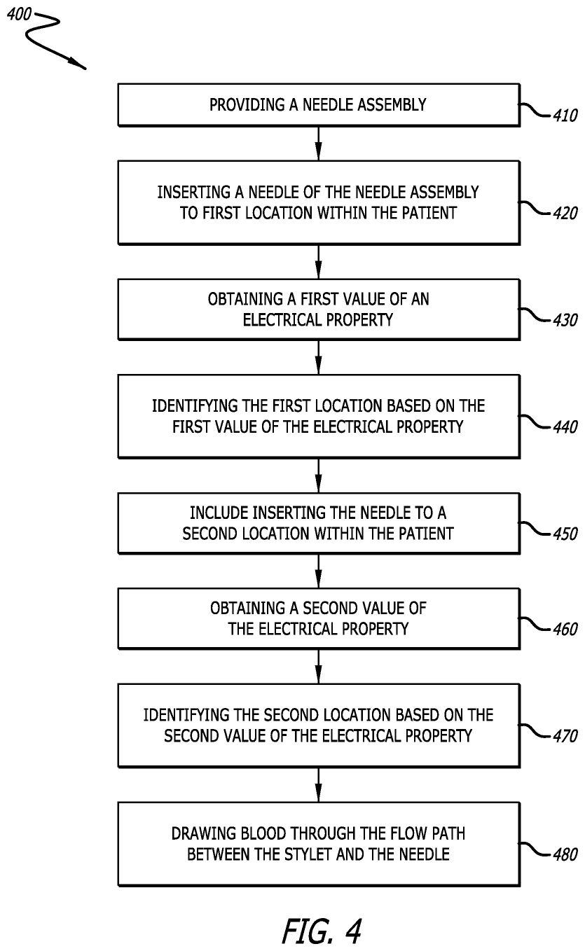

FIG. 4 illustrates a block diagram of an exemplary method 400 of accessing a vasculature of a patient that, according to some embodiments, may include all or any subset of the following steps, actions, or processes. The method 400 may include providing a needle assembly (block 410), where the needle assembly includes a needle configured for vascular access of a patient, and where the needle defines a needle lumen extending between a proximal end and a distal end of the needle. The needle assembly further includes a stylet disposed within the needle lumen, where the stylet includes a non-circular cross-sectional shape. The needle defines a needle electrode, and the stylet includes a stylet electrode adjacent a distal end of the stylet, where the needle and stylet electrodes are configured to enable ascertainment of an electrical property of a body substance adjacent the distal end of the needle. In some embodiments of the method 400, the non-circular cross-sectional shape defines a flow path extending along the needle lumen.

The method 400 may further include inserting the needle to first location within the patient (block 420). The method 400 may further include obtaining a first value of an electrical property (i.e., measuring the first electrical property of the body substance of the first layer) between the needle and stylet electrodes (block 430). The method 400 may further include determining (or identifying) the first location based on the first value of the electrical property (block 440).

In some embodiments of the method 400, the stylet is formed of a stainless steel and the stylet includes an electrically insulative coating extending along a length of the stylet, where the coating defines a uniform thickness.

The method 400 may further include inserting the needle to a second location within the patient (block 450), where the second location is different from the first location. The method 400 may further include obtaining a second value of the electrical property between the needle and stylet electrodes (block 460), where the second value is defined by a second body substance located at the second location. The method 400 may further include determining the second location based on the second value of the electrical property (block 470).

In some embodiments of the method 400, the needle includes a hub, having a fluid port in fluid communication with the flow path and a stylet port configured to receive the stylet therethrough, where the stylet port defines a fluid tight seal with the non-circular shape.

In some embodiments of the method 400, the fluid port is coupled with a syringe via a tube, and the method 400 may further include drawing blood through the flow path between the stylet and the needle (block 480) and into the tube and observing blood within the tube to confirm placement of the needle within the vasculature.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical needle assembly, comprising:
a needle configured for vascular access of a patient, the needle defining a needle lumen extending between a proximal end and a distal end of the needle;
a stylet disposed within the needle lumen, the stylet including a non-circular cross-sectional shape,
wherein:
the needle defines a needle electrode,
the stylet includes a stylet electrode adjacent a distal end of the stylet, and
the needle and stylet electrodes are configured to enable ascertainment of an electrical property of a body substance adjacent the distal end of the needle.

2. The assembly according to claim 1, wherein the stylet is formed of a stainless steel.

3. The assembly according to claim 1, wherein the stylet includes an electrically insulative coating extending along a length of the stylet.

4. The assembly according to claim 3, wherein the coating defines a uniform thickness.

5. The assembly according to claim 1, wherein the non-circular cross-sectional shape extends along an entire length of the stylet.

6. The assembly according to claim 1, wherein a major diameter of the non-circular cross-sectional shape extends across the needle lumen.

7. The assembly according to claim 6, wherein the needle lumen defines:
a first flow path extending along a first side of the non-circular cross-sectional shape, and
a second flow path extending along a second side of the non-circular cross-sectional shape, the second side opposite the first side.

8. The assembly according to claim 6, wherein the non-circular cross-sectional shape includes an oval.

9. The assembly according to claim 1, wherein:
the needle includes a needle lead configured to electrically couple the needle electrode to a measurement module, and
the stylet includes a stylet lead configured to electrically couple the stylet electrode to the measurement module.

10. The assembly according to claim 1, wherein the needle includes a hub, comprising:
a fluid port in fluid communication with at least one of the first or second flow paths, and
a stylet port configured to receive the stylet therethrough, the stylet port defining a fluid tight seal with the non-circular shape.

11. The assembly according to claim 10, wherein:
the stylet is configured for removal from the needle, and
the stylet port includes a septum configured to define a fluid tight seal across the stylet port when the stylet is removed from the needle.

12. A medical stylet, comprising:
a non-circular cross-sectional shape extending along the stylet from a proximal end to a distal end of the stylet,
an electrode adjacent the distal end of the stylet, the electrode configured to electronically couple with a substance of a patient, and
a stylet lead configured to electrically couple the stylet electrode to a measurement module.

13. The stylet according to claim 12, wherein:
the stylet is formed of a stainless steel,
the stylet includes an electrically insulative coating extending along a length of the stylet, and
the coating defines a uniform thickness.

14. The stylet according to claim 12, wherein:
the stylet is configured for insertion within a lumen of a vascular access needle, a major diameter of the non-circular shape extends across the lumen, and
the lumen defines:
a first flow path extending along a first side of the non-circular shape, and
a second flow path extending along a second side of the non-circular shape, the second side opposite the first side.

15. A method of accessing a vasculature of a patient, comprising:
providing a needle assembly, comprising:
a needle configured for vascular access of a patient, the needle defining a needle lumen extending between a proximal end and a distal end of the needle;
a stylet disposed within the needle lumen, the stylet including a non-circular cross-sectional shape,
wherein:
the needle defines a needle electrode,
the stylet includes a stylet electrode adjacent a distal end of the stylet, and
the needle and stylet electrodes are configured to enable ascertainment of an electrical property of a body substance adjacent the distal end of the needle;
inserting the needle to first location within the patient;
obtaining a first value of an electrical property between the needle and stylet electrodes, the first value defined by a first body substance located at the first location; and
identifying the first location based on the first value of the electrical property.

16. The method according to claim 15, wherein:
the stylet is formed of a stainless steel,
the stylet includes an electrically insulative coating extending along a length of the stylet, and
the coating defines a uniform thickness.

17. The method according to claim 16, further comprising:
inserting the needle to a second location within the patient, the second location different from the first location;
obtaining a second value of the electrical property between the needle and stylet electrodes, the second value defined by a second body substance located at the second location; and
identifying the second location based on the second value of the electrical property.

18. The method according to claim 15, wherein the non-circular cross-sectional shape defines a flow path extending along the needle lumen.

19. The method according to claim 18, wherein the needle includes a hub, comprising:
a fluid port in fluid communication with the flow path, and
a stylet port configured to receive the stylet therethrough, the stylet port defining a fluid tight seal with the non-circular shape.

20. The method according to claim 19, wherein the fluid port is coupled with a syringe via a tube, the method further comprising:
drawing blood through the flow path and into the tube; and
observing blood within the tube to confirm placement of the needle within the vasculature.
